(19) United States
(12) Patent Application Publication
(10) Pub. No.: US 2008/0286857 A1
(43) Pub. Date: Nov. 20, 2008

(54) Multi-functional Polymer-Entrapped-Cell-Bead airlift bioreactor for odor or gaseous emission treatment

(75) Inventors: Sz-Chwun John Hwang, Hsinchu City (TW); Hsin-Hua Chen, Hsinchu City (TW); Kuo Shu Huang, Zhunan Town (TW); Jyuhr Yih Lyuu, Kaohsiung City (TW); Yun-Huin Lin, Hsinchu City (TW); Cheng-Ting Hou, Taipie City (TW)

Correspondence Address: TROXELL LAW OFFICE PLLC, 5205 LEESBURG PIKE, SUITE 14044, FALLS CHURCH, VA, 22041, US

(73) Assignee: Chung Hua University

(21) Appl. No.: 12/081,743

(22) Filed: Apr. 21, 2008

(30) Foreign Application Priority Data
May 16, 2007 (TW) 096117480

(51) Int. Cl. C12M 1/34

(52) U.S. Cl. 435/288.6

(57) ABSTRACT

The invention provides a multi-functional polymer-entrapped-cell-bead airlift bioreactor for odor or gaseous emission treatment. Especially, it refers to a reactor that mainly treats a low-and-medium gas flow rate and concentration of volatile organic emission or odorous substances. Particularly, it utilizes synthetic material (PAA beads) and microbial entrapment technology as the microbial source and startup mechanism for gaseous emission and odor treatment. Besides, plastic decomposing Thiosphaera pantotropha can be added under certain condition to be triggered to breakdown the synthetic material (PAA beads) to prevent wastes. This will achieve benefits in low cost, high efficiency and zero secondary pollution.

### BACKGROUND OF THE INVENTION

1. Field of the Invention

The invention is related to a technology to treat organic emission or odors in a medium or low airflow rate, and in a medium or low concentration. Especially, it refers to a multi-functional polymer-entrapped-cell-bead airlift bioreactor that is different from an ordinary bioreactor.

2. Description of the Prior Art

In recent years, the high-tech industry has been through fast development. Many processes in the semiconductor industry (such as microlithography, etching, diffusion, vapor and deposition) and regular equipment maintenance require a huge amount of volatile organic solvent, like acetone, methyl ethyl ketone, isopropyl alcohol, toluene, xylene, chloroform and trichloroethane, et al. If these Volatile Organic Compounds (VOCs) are not carefully handled, they will leak, vaporize or diffuse through chimney to the environment and have adverse influence on the environment and human health. According to the analysis of samples from Hsinchu Science Park (HSP), the main pollutants emitted by the IC industry and the optoelectronic industry are isopropyl alcohol and acetone (50˜80% of total emission quantity). If a physiochemical method is used to treat VOCs, although it is fast, it is not cost effective, and the combustion-exhausted gases, spent scrubbing liquid or wasted activated carbon could cause secondary pollution. It does not contribute to sustainable environment. Therefore, biological treatment by biofilters, bioscrubbers and biotrickling filters has received much great attention in recent years. On a common understanding, biological treatment is very cost effective, and has a very high efficiency in treating odorous or hydrophilic compounds and no secondary pollution concerned. However, over a long period of time it cannot avoid some difficultly operating issues. For examples: for biofilters, the issues include aged problems (filter media decomposition), filter media compression, drying, and a difficulty in pH control; for biotrickling filters, the issues are clogging by air embedded particulates and grown biomass and difficulty for operation; for bioscrubbers, they are not suitable for treating hydrophobic organic pollutants because the hydrophobic compounds has a poor mass transfer efficiency into the water phase of the water-in-air system, and other issues including easy clogging in sprayer to cause uneven spray and requiring secondary treatment for sludge after absorbing VOCs.

In the early time, the main applications for airlift bioreactor were in synthesis of organics, wastewater treatment and fermentation (for beers, vinegar, and citric acid). There is no literature in the past about using synthetic materials to entrap specific cell beads for airlift bioreactor to treat VOCs from stationary pollution emission. It was known that an airlift bioreactor was used to inject gaseous pollutants from the bottom of the reactor, and through a good gas-liquid mixing and absorption, the pollutants were mixed into the liquid phase in the reactor and then subject to microbial decomposition. But it only used activated sludge as cultural source and did not add artificial polymer entrapped cell beads with specific microorganisms which the inventor want it to be. Some literatures indicate the use of charcoal absorbed activated sludge to keep the microorganisms on the surface or combinational use of airlift reactor and others.

On the other hand, a low concentration and large airflow rate are the typical characteristics of gaseous VOC emission by the semiconductor industry. Currently companies in HSP mostly use rotor concentrators to work with thermal oxidizer. The airflow entering the thermal oxidizer has been greatly reduced by the rotor concentrator, so now the airlift reactor can replace the high-operating-cost thermal oxidizer. The emission from the rotor concentrator has fairly a high concentration of pollutants. It could harm the suspended microbes in the conventional reactor or inhibit their growth. Nevertheless, many researches found that immobilization technology has the advantages in protecting the microbes, high operational stability and preventing loss of microbes, et al. This invention provides a solution to the above issues.

The conventional technology has the following drawbacks:

1. Common drawbacks occurring for the conventional biological treatment of gaseous emission include a low treatment efficiency in a high influent concentration, undesirable environment factors (like temperature, pH value, ionic strength et al:) and presence of toxic substances, easy wash-out, extra care for microbial growth, and no controllable manner to provide a specific microorganism.

2. Biofilters:

Its issues include aged problems (filter media decomposition), filter media compression, drying and a difficulty in pH control, et al.

3. Biotrickling Filter:

The pore of packing materials tends to have a clogging problem due to massive population from microbial growth. Unclogging is impossible once it is clogged. It requires extra nutrition sources that add cost.

4. Bioscrubbers:

First, it is a pure water-in-air system. Since the hydrophobic pollutants prefer to exist in the gas phase, it is very difficult to capture them with water droplets. For mass transfer efficiency, this is the worst in the three conventional biological treatment methods for emission. Second, there is a clogging issue with water sprayer that causes uneven spray. To solve the clogging problem, usually the upper supernatant is screened and drawn back to the spray nozzle. This certainly fails to capture hydrophobic materials without the slurry (liquid and solid phase). Finally, little amount of VOCs were adsorbed into the liquid, which is then subject to activated sludge degradation.

5. Aeration in Activated Sludge:

The activated sludge method is limited by the large axial dispersion effect: under certain height many hydrophobic pollutants with dimensionless Henry's constant above 0.1 cannot be completely absorbed.

6. Conventional Airlift Bioreactor:

Their conventional applications are mainly for fermentation or pharmaceutical manufacturing. For environmental engineering, it is sometime used in wastewater treatment. However, the airlift reactor with artificial polymer-immobilized cell beads has never been adopted to treat air pollutants in gaseous emission.

Because the conventional technologies and equipments to treat odors or gaseous emission still have drawbacks that are undesirable and need improvement, the inventor spent many years experiencing in the related researches with professional expertise to conduct many long-term experiments and testing, and finally invented this new technology that is expected to benefit the public.

### SUMMARY OF THE INVENTION

The main objective for the invention is to provide a new multi-functional artificial polymer-immobilized cell-bead airlift bioreactor for odor or gaseous volatile organic compound (VOC) emission treatment that attenuates the influences of the drawbacks found in previously mentioned various biofilter technologies or the activated sludge method. It is expected to completely solve the clogging problem of the filtering bed and achieve improved gas-liquid mixing effect. It will be enhanced for gas pollutants being absorbed in liquid phase due to the adsorption by the associated cell beads, and the cell beads can be broken down by certain suspended microbes when the denitrification is triggered as the inventor requires.

Another objective for the invention is to provide new multi-functional bead airlift bioreactor for odor or gaseous emission treatment that adopts a lower diameter-to-height ratio configuration than a conventional air treatment bioreactor, and requires less space area for installation especially good for the factories which can not provide more space for doing air pollution control.

To achieve the above objectives, the inventor in the invention has two added designs as follows:

(1) In the riser there are many PVC pipes with the specified diameters or there are several inserted panels to form compartments in grid.

(2) For different pollutants, a specific microbial strain is selected to be entrapped with the polymer to form a cell bead that will fill into the airlift reactor. Using the entrapped-microbes for start up (a manner could retain much more cell concentrations for use) could quickly initiate the decomposition of VOCs. The invention can be modified according to different needs. If it is used for system startup, different entrapment methods (like PAA system) can be adopted with added plastic-decomposing Thiosphaera pantotropha in certain concentration of ammonia-nitrogen or nitric-acid-nitrogen. Through denitrification, PAA-immobilized cell beads can be used as carbons source and electron provider for further decomposition without the needs of considering wasted PAA treatment.

The application scope for the invention is to treat VOCs or odors in medium and low airflow and in medium and low concentration. Classification can be made by organics and inorganics, or by the industrial type applied. Presently, the known organics or inorganics that can be treated by microbes include various carbonaceous volatile organic compounds (CVOCs), simple volatile sulfuric organic sulfides (SVOCs), volatile nitric organic nitrides (NVOCs), and odorous substances like inorganic ammonia-nitrogen and hydrogen sulfide. The industries that directly emit VOCs from their processes include electronics industry, surface coating industry, printing industry, oil industry, agrochemical manufacturing, leather industry, food industry and semiconductor industry. This industrial category also applies to those that use solvents or odors emitted by secondary collection and exhaust system, sewage treatment plant and garbage landfill sites.

Preferred embodiments with figures will be described in the following for patent examiners to further understand the invention.

### BRIEF DESCRIPTION OF THE DRAWINGS

FIG. 1 is the system structure diagram for a preferred embodiment of the airlift bioreactor in the present invention.

FIG. 2 is the exterior and crossection structure diagram for a preferred embodiment in the present invention.

FIG. 3 is the flow diagram for PAA immobilization for a preferred embodiment in the present invention.

### DETAILED DESCRIPTION OF THE PREFERRED EMBODIMENT

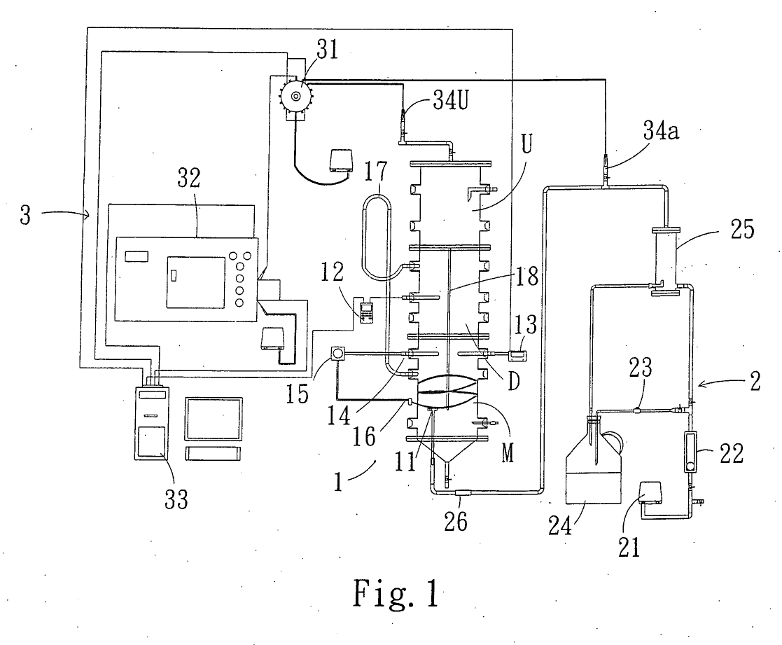

Please refer to FIG. 1 for the airlift bioreactor system structure in a preferred embodiment of the invention, which consists of:

an airlift reactor (1), an aerator (11), a D.O. meter (dissolved oxygen electrode) (12), a pH meter (acid-base electrode) (13), a temperature sensor (14), a temperature controller (15), heating tape (16), a U-Tube manometer (17), a riser (18). The airlift reactor (1) is made of acrylics with 41 L working volume and inner diameter 19 cm. It is operated by a counter current flow mode that pumps the clean air through the pipes passing the VOCs (acetone) make-up system (2) and blowing up from the bottom of the airlift reactor (1). The sparger (11) is a trapezoidal gas distributor and placed in the lower section of the reactor. On the two sides of the airlift reactor (1) a D.O. meter (dissolved oxygen electrode) (12) and a pH meter (acid-base electrode) (13) are installed in the middle for monitoring and control. The PAA-immobilized cell beads are about 1 L and filled into the reactor (1). The gas will force the liquid (water) through the riser (18) up to the upper complete mixing zone (U), the downcomer (D) and the lower complete mixing zone (M) in a cyclic way. Simultaneously, the cell beads with specific gravity slightly less than 1.0 will move with the flow and evenly distribute themselves everywhere in the pipe to carry out VOC decomposition. Additionally, active carbons can be put in the reactor (1) to capture benzene and surfactants can be use to improve the surface hydrophobic characteristics for VOCs passing into the cell beads.

There is also a VOCs (Volatile Organic Compounds) supply system (2) that consists of an electromagnetic diaphragm pump (21), a rotameter (22), a needle valve (23), a VOC storage bottle (24), an equalization column (25), and a check valve (26). Clean air is first generated by sucking the outdoor air through a filtration device by the electromagnetic diaphragm pump (21). After flowing through a rotameter (22) and a needle valve (23), the clean air enters the VOC bottle (24) that contains a diluted acetone solution to generate the VOC-containing (acetone) gas. Finally, it flows into an equalization column (25) and is evenly mixed with the other route of clean air generated by the same electromagnetic diaphragm pump (21). The acetone concentration can be adjusted through the needle valve (23) by controlling the gas flow rate. Finally, the VOC (acetone)-containing air from the equalization column (25) flows into the check valve (26), and to the entrance at the lower section of the airlift reactor (1) for treatment.

There is also an automatic sampling and analysis device (3) that consists of an auto sampler (31), a GC (Gas Chromatography) (32), and a computer (33). In the outlet of the equalization column (25) (influent concentration measurement) and the airlift reactor (1) (effluent concentration measurement) there are two sampling stainless steel tubes (34a, 34b) that connect to the auto sampler (31) where it has 16 sampling ports available for more influent gas and treated effluent gas streams. When gas sampling is underway, through computer (33) program execution, the sampling ports of the auto sampler (31) will switch in order, so the sampling point corresponding to the sampling port is connectable through the steel tubing into the GC (Gas Chromatography) (32) for analysis. The unspecified gas collected by the rest of sampling ports will be exhausted out of the auto sampler (31). The analogue voltage signal associated with peak and valley voltages obtained from the GC (Gas Chromatography) (32) signal output is converted through a data acquisition card into digital signal, and then the data are stored in the computer (33) program for further data processing. At the end of the downcomer (D) of the airlift reactor (1) liquid sample is collected to analyze acetone concentration or other components in the aqueous phase.

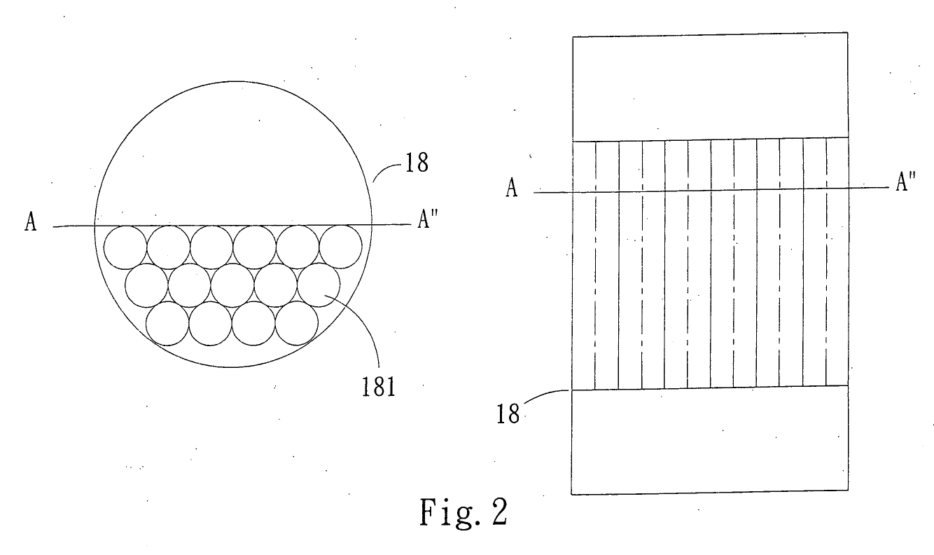

Please refer to the exterior diagram and crossection structure diagram for the riser of the airlift reactor in FIG. 2 for the features of the invention. It shows PVC pipes (181) with specified diameters is added in the riser (18) of the airlift reactor (1), or separating panels are used to make grid to prevent large bubble formation in the riser (18). Small bubbles confined by the PVC pipes (181) or grid separation can increase gas holdup and, in other words, the retention time of gaseous pollutants.

The main feature for the invention is to use artificially entrapped-cell beads as reactor microbial carrier and use entrapped-cell for a fast startup. If there is a shock loading, the microbes is protected by the barrier of the beads and able to continuously retain the necessary amount of microbes. The artificial carrier is durable. If the cell bead is used for system startup, it only needs to add the polymer-decomposing strain, Thiosphaera pantotropha triggered by a certain concentration of ammonia nitrogen. Through denitrification, PAA polymer will be broken down. The preparation method for polyacrylamide (PAA) is briefly described as follows:

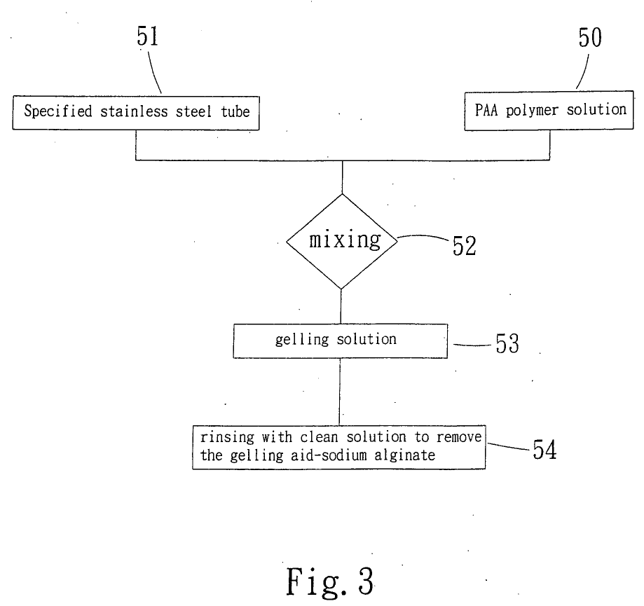

Please refer to FIG. 3 for the process flow of PAA immobilization, which includes preparing the PAA polymer solution (50); preparing a specific microbial culture or mixed culture (51); mixing (52); preparing a gelling solution (53); and rinsing with clean solution to remove the gelling aid-sodium alginate (54). In a practical operation, AA monomers, crosslinking agent (BIS), accelerator (TEMED), sodium alginate (0.5%, w/v) and active carbons powders and surfactants (for treating hydrophobic pollutant) are mixed in a certain ratio and added into 4° C. sterile distilled water to complete the preparation of PAA polymer solution. Next, the pump injects the PAA polymer solution and concentrated microbial solution into the small syringe space for fast mixing. The solution is then dropped into a gelling solution that contains calcium chloride (3%, w/v) and ammonium persulfate (0.5%, w/v) under agitation. The sodium alginate in the gelling solution will speed up the formation of polymer beads. After 3 hours of polymerization, the PAA-immobilized cell beads are transferred to potassium phosphate buffer solution (pH 7.8) for agitation for one hour. So the sodium alginate in the PAA polymer beads can be dissolved and washed out. At the same time, the size of PAA-immobilized cell beads can be controlled by the syringe needle size in the immobilization process.

The main principle for the invention is to utilize the enhanced absorption capability of the water by microbial entrapped-cell beads to capture the pollutants into the liquid phase, and then further transfer the pollutants into the beads, so the microbes will undergo metabolism and decompose the pollutants. Thus, there are the following advantages:

1. The entrapment method for the airlift reactor in the present invention is to protect the microbes in artificial polymer materials, different than biofilter and biotrickling filters that the microbes directly adhere to the biofilm on the filter surface, so it is fairly difficult for poisoning to happen. It also enables influent with high or shock mass load or abrupt change of environmental influencing factors. Besides, its exterior protection layer pi-events competition among different microbial species. Certainly, it will not be inhibited due to system overload.

2. For biofilters, since absorbing solution is liquid and flowable, it does not have clogging issue. Also because liquid is flowable, pollutant transfer is relatively faster by the fast surface renew and agitation uniformity is attainable. It is not like in filtering bed biofilm that slow diffusion is the only necessary mechanism. It will be easier for gaseous pollutants to be uniformly absorbed in the liquid phase and enter the beads for diffusion and metabolism. Besides, the greatest advantage is the reactor type can be long high pillar shape, which occupies less space than the biofilter.

3. For biotrickling filter, because the absorbing solution and the polymer-immobilized cell beads are uniformly mixed, nutrition from dead microbes after decomposition is easier to be completely released. So there is no need of additional nutrition after the system is stabilized. It will also not have clogging problems.

4. For bioscrubbers, although it adopts the same absorption principle, the airlift reactor in the present invention has better mass transfer efficiency because the reactor adopts the air-in-water mechanism that the pollutants are contained in small air bubbles. The airlift reactor does not need spray nozzle to spray absorbing solution, so the microbial floc can be mixed with the absorbing solution to form slurry that will directly decompose the pollutants without the need of aeration tank for activated sludge. Since the cell membrane for the microbes in the slurry is made of phospholipids, an amphilic substance, it will have enhanced absorbing ability for hydrophobic materials.

5. For activated sludge aeration method, since the riser in the reactor can be compartmented, selecting suitable grid size based on hydrophobicity of pollutants, the gas retention rate for hydrophobic materials can be properly controlled.

6. For conventional airlift bioreactors,.they are mainly compared upon the three-phase reactor. The three phases are air, absorbing solution and suspended microbial solid. Since the conventional airlift reactor needs filling activated sludge before treating pollutants, if the activated sludge contains VOCs, they will be carried out by gas. Thus, it creates another pollution issue before solving a pollution issue. If no activated sludge is used, there will be no source of microbes, which is equivalent to absorbing with clean water and produces no effect for the long term. On the other hand, low concentration and large airflow are the typical characteristics of VOC emission by the semiconductor industry. Currently companies in HSP mostly use rotor concentrators to work with thermal oxidizer. The airflow entering the thermal oxidizer has been greatly reduced by the rotor concentrator, so airlift reactor can replace high-cost thermal oxidizer. The emission from the rotor concentrator has fairly high concentration. But with immobilization technology to protect the microbes, which will have high stability and be prevented from loss, the invention actually has broader application scope.

In summary, the Multi-functional Polymer-Entrapped-Cell-Bead Airlift Bioreactor for Odor or Gaseous Emission Treatment in the present invention not only has innovative structure but also new type of carrier in the reactor. It will achieve the performance than could have not been attained before. So it shall meet the requirements for patent application.

What is claimed is:

1. A multi-functional polymer-entrapped-cell-bead airlift bioreactor for odor or gaseous emission treatment consists of
an airlift reactor so its lower section has a sparger, its tank has a D.O. meter (dissolved oxygen electrode) and a pH meter (acid-base electrode) on two sides of the middle section, there is a riser in the center and there are also upper mixing zone (U), downcomer (D) and lower mixing zone (M);
a VOCs (Volatile Organic Compounds) supply system that consists of an electromagnetic diaphragm pump, a rotameter, a needle valve, a VOC bottle, an equalization column, a check valve, and an entrance for VOCs (acetone) vapor and mixture of VOCs (acetone) and air to enter the lower section of the reactor;
an automatic sampling device that consists of an auto sampler, a GC (Gas Chromatography) and a computer, so at the outlet of the equalization column (influent concentration measurement) and the airlift reactor (effluent concentration measurement) there are sampling steel tubes connecting to the auto sampler to collect the influent gas and the treated effluent gas for analysis with features that:
in the riser there add PVC pipes with different diameters to prevent large bubble formation inside the tube, so the smaller bubbles separated by the PVC pipes can increase gas holdup and gas retention time.

2. As described in claim 1 for a multi-functional polymer-entrapped-cell-bead airlift bioreactor for odor or gaseous emission treatment, separation inside the riser can be made by panels to form grid of compartments.

3. As described in claim 1 for a multi-functional polymer-entrapped-cell-bead airlift bioreactor for odor or gaseous emission treatment, the reactor has carrier such as artificially polymer-entrapped cell beads, which serves as a fast system startup manner to supply microbes to decompose VOCs.

4. As described in claim 3 for reactor carrier, the carrier can be polyacrylamide (PAA).

5. As described in claim 3 for reactor carrier, the synthetic carrier can be added with plastic decomposing Thiosphaera pantotropha to breakdown PAA.

6. As described in claim 3 for reactor carrier, the immobilization process for the artificial synthetic carrier includes preparation of PAA polymers solution, preparation of specific microbial culture solution, mixing, preparing a gelling solution and rinsing with cleaning solution.

7. As described in claim 1 for a multi-functional polymer-entrapped-cell-bead airlift bioreactor for odor or gaseous emission treatment, activated carbon is added inside of the airlift reactor to capture hydrophobic pollutants and with surfactants to help VOCs passing through the channels inside the cell beads.
